(19) United States
(12) Patent Application Publication
(10) Pub. No.: US 2016/0317661 A1
(43) Pub. Date: Nov. 3, 2016

(54) DERMAL AND/OR TRANSDERMAL PHARMACEUTICAL COMPOSITION CONTAINING A TERPENOID COMPOUND

(71) Applicant: EPINAMICS GMBH, Berlin (DE)

(72) Inventors: Hans-Michael THIEDE, Postdam (DE); Wolfgang KEHR, Berlin (DE)

(73) Assignee: EPINAMICS GMBH, Berlin (DE)

(21) Appl. No.: 15/104,720

(22) PCT Filed: Aug. 22, 2014
(86) PCT No.: PCT/DE2014/000420

§ 371 Date: Jun. 15, 2016

(30) Foreign Application Priority Data
Dec. 16, 2013 (DE) IO 2013 020 633.2

(51) Int. Cl. A61K 47/10; A61K 9/70; A61K 31/568; A61K 9/00

(52) U.S. Cl. CPC A61K 47/10; A61K 9/0014; A61K 9/7015; A61K 31/568

(57) ABSTRACT

The invention relates to a method of solubilizing a pharmaceutical active ingredient, to the use of this method of making a pharmaceutical composition in the form of a dermal and/or transdermal preparation, to a pharmaceutical composition containing this compound, and to at least one pharmaceutical active ingredient, an aerosol dispenser with such a composition, and to a method of making such a composition.

### FIELD OF THE INVENTION

The invention relates to a method of solubilizing a pharmaceutical composition and to the use of such a method or a compound for making a pharmaceutical composition in the form of a dermal or transdermal preparation, wherein a pharmaceutical active ingredient is solubilized in a pharmacologically effective amount in the compound. The invention further relates to pharmaceutical compositions containing such a compound, to an aerosol dispenser containing such a composition, and to a method of making such a composition.

### BACKGROUND OF THE INVENTION AND PRIOR ART

Another form of administration of compositions of active ingredients, besides, for instance, the oral administration, the inhalation, or injection, comprises the so-called transdermal systems. Herein, the composition of active ingredients is applied on the skin of a human or an animal, and depending on the configuration of the transdermal system, is resorbed more or less quickly. The active ingredients may act locally or systemically. The advantages of the transdermal administration over the inhalation or also the oral administration include a better compatibility, a localized active ingredient targeting, controlled resorption rates, and thus the avoidance of reduced bioavailability of the active ingredient.

Transdermal systems may, for instance, be designed as adhesive patches, so basically they consist of a patch to be applied on the skin, comprising a substrate, in or on which the composition of active ingredients is arranged, and comprising an adhesive layer, through which the composition of active ingredients reaches the skin by way of permeation and is resorbed there. An example is a nicotine patch according to WO 2000/033812 A. Therein, the transdermal preparation is applied or sprayed on a transfer film. Then typically follows a coating of the dried transdermal preparation comprising an adhesive layer with a skin-compatible adhesive. Thus, the patch can be placed with its side facing away from the transfer film on the skin, and can be adhered there. Such patches are disadvantageous, since they are optically disturbing (they often cover large areas for the purpose of making a sufficient amount of active ingredient available for the transdermal release), and may further cause problems with regard to water resistance and the safe adhesive attachment on the skin, with the consequence of reduced active ingredient absorption by the patient, in particular the consequence of impaired controllability of the bioavailability of the active ingredient over the given period of treatment.

As patches, however, are also referred to coatings on the skin, which are sprayed as an aerosol on a target area of the skin and form an adhering coating on the target area. Typically, the aerosol compositions contain a film-forming polymer as a matrix, which forms the actual patch, and a composition of active ingredients, which during the spraying process on the skin is embedded in this matrix. The polymer of the matrix is selected and adapted such that the delivery rate of the active ingredient to the skin is adjusted to a set-point value. In most cases, the aerosol composition also comprises at least one solvent, typically a slightly volatile, pharmacologically acceptable organic solvent being compatible with the composition of active ingredients and with the polymer. Sometimes, the aerosol also contains a propellant, for instance when the aerosol dispenser is configured as a spray bottle. The propellant may also act as a solvent. An example of a composition suitable for spraying for the purpose of forming a patch is described in the document WO 2001/037890.

A disadvantage of such transdermal compositions is that very many active ingredients are poorly or not at all soluble in the solvents used for the solutions. Therefore, the patch, irrespective of whether produced on a transfer film, or generated directly on the skin by way of spraying, will contain a rather low amount of active ingredient. Then, high doses cannot be administered transdermally at all. Further, the patches unnecessarily have to be prepared as larges areas.

The above explanations analogously also apply to dermal applications, i.e., when the contained active ingredient takes its full effect at the site of application.

The use of compounds, such as carvacrol or thymol, as a biocide, as a drug inhibiting inflammations, as an antibiotic or antimycotic is known from practical experiences, see, for instance, de.wikipedia.org/wiki/carvacrol.

### Technical Object of the Invention

It is therefore the technical object of the invention to provide means, by means of which compositions for making patches can be prepared that have, compared to prior art, an increased concentration of the active ingredient. It is further the object of the invention to provide means, which allow the production of small patches, compared to prior art, with an unchanged amount of active ingredient content (absolute).

### BASICS OF THE INVENTION AND PREFERRED EMBODIMENTS

For achieving this technical object, the invention teaches a method of solubilizing a pharmaceutical active ingredient in an aqueous and/or organic solvent, the solvent being a compound of Formula I or a mixture of different such compounds, or a mixture of the compound or of the mixture of the different such compounds with an additional solvent, the active ingredient being solubilized in the compound or in the solvent containing the compound,

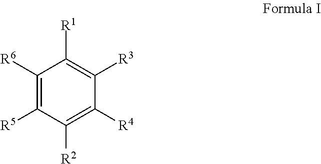

wherein R1 to R5 are independently from each other, identically or different, selected from the group consisting of —H, —OH, methyl, ethyl, propyl, isopropyl, isopropenyl, and alkoxy, wherein R6 is selected from —O—R7 and —CO—R7, with R7 being selected from —H, C1-C3-alkyl, C6-aryl and C7-C10-aralkyl, substituted or not substituted, and and wherein the aromatic ring of the illustration of Formula I may be replaced by C6-cycloalkyl, saturated or mono-unsaturated or di-unsaturated, at an arbitrary position of the cycloalkyl ring.

It is preferred, with R6=—OH, that R1 is methyl or methoxy, R2 is isopropyl, isopropenyl, or —H, and R3 to R5 are —H, or R1 is isopropyl, R2 is methyl, and R3 to R5 are —H. Also is preferred that R6=—OCH3 or —CO—CH3, with at least one (arbitrary) residue R1 to R5 as a methyl.

The invention teaches in particular the use of such a method of making a pharmaceutical composition in the form of a dermal or transdermal preparation, wherein a pharmaceutical active ingredient in pharmacologically effective amount is solubilized in the compound.

Alkoxy comprises, herein, in particular alkyl residues with 1, 2, 3, 4, or 5 C atoms, linear or branched.

The invention is based on the surprising finding that very many pharmaceutical active ingredients can be solubilized in a considerably better way in such compounds, than in solvents used up to now for dermal or transdermal systems. That is, in this invention, the compound used according to the invention does not represent an active ingredient, but basically acts as a solvent and, if applicable, in addition as a galenic means mediating penetration. It follows that with a preparation according to the invention, dermal and/or transdermal systems can be made, which either contain significantly larger amounts of active ingredients, can be formed with smaller patches than previously, or both. Further, by a synergistic effect, a larger proportion of compound of Formula I has the effect of a particularly effective penetration enhancement, i.e., the passage of the pharmaceutical active ingredient through the skin and into the body is improved.

In addition to the compound of Formula I with the active ingredient solubilized therein, a dermal and/or transdermal preparation typically, but not necessarily, comprises at least one film-forming polymer. If no such polymer is provided, the preparation will not form a patch after application on the skin, rather the active ingredient is immediately resorbed in the skin. After application of the preparation on the skin or on a transfer film, the compound will evaporate, and a film is formed from the polymer, in which the active ingredient is embedded and is released in a controlled manner. For this purpose, typically, active ingredient and polymer are matched to each other, in order to secure predefined release rates to the skin. For the purpose of the invention, the selection of the polymer is however in principle irrelevant, since it may either be solubilized in the compound, or in the case of poor solubility, emulgated or suspended therein. In a preparation according to the invention, in addition to the compound of Formula I used according to the invention, another conventional additional organic solvent may also be employed, for instance for improving the solubilization of the polymer and/or for reducing the volatility of the compound of Formula I.

In the following, preferred embodiments of the invention are described.

The pharmaceutical composition may contain the following components: a) at least one compound of Formula I or a mixture of different such compounds, b) at least one dermally acting and/or transdermally resorbing pharmaceutical active ingredient or a mixture of different such active ingredients, c) optionally at least one film-forming polymer, d) optionally at least one aqueous or additional organic solvent or a mixture of different such additional solvents (then it is a solution of the active ingredient or the active ingredients in a mixture of the compound of Formula I and of the additional solvent or additional solvents), e) optionally at least one galenic excipient or a mixture of different such excipients, f) optionally at least one dermo-cosmetically acting additive or a mixture of different such additives. The use of at least one additional solvent is recommended in particular in those cases, where the compound of Formula I at room temperature (20° C.) is not liquid or is solid.

Typically, the preparation contains at least 1 wt.-%, 5 wt.-%, or 10 wt.-%, for instance at least 20 wt.-%, 30 wt.-%, 40 wt.-%, 50 wt.-%, 60 wt.-%, 70 wt.-%, 80 wt.-%, or 90 wt.-% of the compound of Formula I (referred to the total amount of freshly produced preparation). In a conventional manner, an amount of the active ingredient is solubilized therein that satisfies the desired pharmacological efficiency. The further components are contained in conventional amounts in the preparation. This includes, for instance, 2 to 30 wt.-%, in particular 5 to 20 wt.-%, preferably 5 to 10 wt.-% polymer, wherein the remainder to achieve 100 wt.-% is then formed by active ingredient or active ingredients and the further optional components mentioned elsewhere, such as in particular additional solvents.

An additional organic solvent being used, if applicable, and being different from the compound of Formula I is typically a physiologically compatible organic or aqueous additional solvent. These include, for instance, organic solvents, such as benzyl alcohol, ethanol, methanol, butanol, isobutanol, diacetone alcohol, methylene chlorides, carbon tetrachloride, trichloroethylene, chlorothene, ethyl acetate, n-propyl acetates, n-butyl acetates, isobutyl acetate, amyl acetate, duPont DBE, Exxate 500, 700, 900, glycol, and ether/ester derivatives, ethylene glycol, PM-acetate, butyl-celluosolve, carbritol acetate, butylcarbritol acetate, Ektapro EEP, toluol, Xylol, VM&P naphtha, mineral solvents, Arouratic 100, Aromatic 150, acetone, methyl ethyl ketone, methyl butyl ketone, dimethyl ether, benzyl benzoate, isopropyl myristate, acetoneitril, ethyl-oleate, glycerol, glycofurol (tetraglycol), propylene glycol, polyethylene glycol (PEG), hexane, n-hexane, glycol ether, methylene chloride, methyl chloride, octyldodecanol, trichloroethane diethyl phthalate, and dibutyl phthalate. Preferably, the solvent is selected from the group consisting of methanol, ethanol, butanol, 1-propanol, 2-propanol, 1-bromopropane, 1-octanol, dimethyl ether, diethyl ether, methyl ethyl ether, benzene, chloroform, DMSO, acetoneitril, acetone, dioxane, ethylene glycol, propylene glycol, dimethyl formamides, benzene, ethyl acetate, PEG400, hexane, trichloroethylene, ethyl catate, N-methyl-2-pyrrolidone, toluene, DMSO, DMF, pyridine, isopropyl myristate, dichloromethane, 1,4-dioxane, and aniline.

Preferred is the use of at least one additional solvent, which reduces the volatility of the compound of Formula I, i.e., arranges for that this compound will as slowly as possible escape from the applied preparation or from the patch, if applicable, in a mixture with other additional solvents, which per se will not reduce the volatility of the compound of Formula I. In so far, the additional solvent can also act as an agent reducing the volatility with regard to the compound of Formula I. This is advantageous, since thereby, on the one hand, an undesired crystallization of the active ingredient (basically existing in a high concentration, due to the compound of Formula I) in the preparation or the sprayed patch is prevented, and, on the other hand, the penetration-mediating effect (with respect to the penetration of the active ingredient through the skin) of the compound of Formula I will be preserved for a long time. Examples of preferred additional solvents include therefore propylene carbonate, propylene glycol, and isopropyl myristate. According to the procedure of Example 2, those skilled in the art will easily identify other in so far particularly suitable additional solvents.

A reduction of the volatility refers to a volatility of the compound in the solvent with the additional solvent, which is reduced by at least 5%, preferably at least 10%, most preferably at least 20%, in particular at least 50% relative to the volatility of the compound in a solvent without the additional solvent.

When the additional solvent is aqueous or when it is water or contains water, it is recommended to adjust the pH by conventional additives to a range from 4.0 to 9.5, preferably 6.0 to 9.0, in particular 6.0 to 8.0.

The proportion (referred to mass) between solvent and additional solvent, if an additional solvent is employed, may be between 1:20 and 1,000:1, in particular between 1:10 and 10:1, for instance between 1:5 or 1:4 and 10:1. The upper limit of the range is only defined by potential traces of an additional solvent, for instance from the admixture of other components of a preparation. A particularly interesting finding of the invention is that with such mixtures often a considerable synergistic effect can be observed, i.e., the solubility of the active ingredient in the mixture is considerably higher than in the solvent or the additional solvent alone. This applies, for instance, in particular to ethanol as an additional solvent.

The melting point of a solvent used according to the invention may in so far be critical, as without using an additional solvent, the solvent should be liquid at room temperature (20° C.). Preferably the melting point is below 10° C., for instance below 0° C.

When using an additional solvent, the melting point of the solvent may however also be above room temperature. Preferably, the melting point is then below 150° C., for instance below 120° C., in particular below 100° C., or below 80° C., 60° C., or 50° C. Interestingly, the effects according to the invention are also then achieved, when a solvent being solid at room temperature is first solubilized in an additional solvent, before the active ingredient is solubilized in the mixture of solvent and additional solvent.

The invention is particularly well suited for pharmaceutical compositions for treating bacterial inflammations, fungal infections, in particular onychomycosis (here, too, the high penetration enhancement of the compound of Formula I shows advantages).

Examples of dermal and/or transdermally acting pharmaceutical active ingredients used according to the invention include: antidiarrheals, such as diphenoxylates, loperamides, and hyoscyamines; cardiovasculars, such as hydralazines, minoxidil, captopril, enalapril, ramipril, clonidin, prazosin, debrisoquine, diazoxide, guanethidine, methyldopa, reserpine, trimetaphan, diltiazem, felodopine, amlodipine, nitrendipine, nifedipine, and verapamil, amiodarone, flecainide, disopyramides, procainamides, mexiletene, quinidine, glyceryltrinitrate, erythritol tetranitrate, pentaerythritol tetranitrate, mannitol hexanitrate, perhexilene, isosorbide dinitrate nicorandil, alprenolol, atenolol, bupranolol, carteolol, labetalol, metoprolol, nadolol, nadoxolol, oxprenolol, pindolol, propranolol, sotalol, timolol, and timololmaleate, digoxin, adrenaline, ephedrine, fenoterol, isoprenaline, orciprenaline, rimeterol, salbutamol, salmeterol, terbutaline, dobutambe, phenylephrine, phenyl propanolamine, pseudoephedrine, dopamine, cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate, xanthinol, ergotamine, dihydroergotamine, methysergide, pizotifen, and sumatriptan; anticoagulants, and thrombolytic agents, such as warfarin, dicoumarol, low molecular weight heparine, such as noxaparin, plasminogen activators, such as streptokinase, t-pA, and haemostatic agents, such as aprotinin, tranexamic acid, and protamine; ZNS agents, such as buprenorphine, dextromoramides, dextropropoxyphene, fentanyl, alfentanil, sufentarlil, hydromorphone, methadone, morphine, oxycodone, papaveretum, pentazocine, pethidine, phenoperidine, codein, dihydrocodein, acetylsalicylic acid (ASS), paracetamol, phenazone, barbiturate, amylobarbitone, butobarbitone, pentobarbitone, choralhydrate, chloromethiazole, hydroxyzine, meprobamate, benzodiazepine, 1prazolam, bromazepam, chlorodiazepoxide, clobazam, chloroazepate, diazepam, flunitrazepam, flurazepam, lorazepam, nitrazepam, oxazepam, temazepam, triazolam, phenothiazines, chloropromazine, fluphenazine, pericyazine, perphenazine, promazine, thiopropazate, thioridazine, and trifluoperazine, butyrophenone, droperidol, haloperidol, pimozide, thiothixene, lithium, tricyclic antidepressants, amitryptyline, clomipramine, desipramine, dothiepin, doxepin, imipramine, nortriptytine, opipramol, protriptyline, trimipramine, tetracyclic antidepressants, mianserin, monoaminoxidase inhibitors, isocarboxazid, phenelizine, tranylcypromine, moclobemide, fluoxetine, paroxetine, citalopram, fluvoxamine, sertraline, coffein, tacrine, amantadine, benserazide, carbidopa, levodopa, benztropine, biperiden, benzhexol, procyclidine, s(-)-2-(N-propyl-N-2-thienylethylamino)-5-hydroxytetralin (N-0923), phenytoin, valproic acid, primidone, phenobarbitone, methyl phenobarbitone, carbamazepine, ethosuximide, methsuximide, phensuximide, sulthiame, clonazepam, phenothiazines, prochloperazine, thiethyl perazine, ondansetron, granisetron, dimenhydrinate, diphenhydramine, metoclopramides, domperidone, hyoscine, hyoscine hydrobromide, hyoscine hydrochloride, clebopride, compride; anti-inflammatory agents, such as ibuprofen, flurbiprofen, ketoprofen, aclofenac, diclofenac, aloxiprin, aproxen, diflunisal, fenoprofen, indomethacin, mefenarnic acid, naproxen, phenylbutazone, piroxicam, salicylamides, salicylic acid, sulindac, desoxysulindac, tenoxicam, tramadol, ketoralac, flufenisal, salsalate, triethanolamin salicylates, aminopyrine, antipyrine, oxyphenbutazone, apazone, cintazone, flufenamic acid, clonixeril, clonixin, meclofenarnic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamides, letimide hydrochloride, nexeridine hydrochloride, octazamides, molinazole, neocinchophen, nimazole, proxazolecitrate, tesicam, tesimide, tolmetin, triflumidale, penicillamine, aurothioglucose, sodium aurothiomalate, methotrexate, auranofm, baclofen, diazepam, cyclobenzaprine hydrochloride, dantrolene, methocarbamol, orphenadrine, quinine, allopurinol, colchicine, probenecid, and sulphinpyrazone; hormones, steroids, and steroidal substances, such as oestradiol, oestriol, oestrone, ethinyloestradiol, mestranol, stilboestrol, dienoestrol, epioestriol, estropipate, and zeranol, allyloestrenol, dydrogesterone, lynoestrenol, norgestrel, norethyndrel, norethisterone, norethisterone acetates, gestodene, levonorgestrel, medroxyprogesterone, megestrole, cyproterone acetates, danazol, epitiostanol, exemestane, 4-hydroxy-androstenedione, testosterone, methyl testosterone, clostebol acetates, drostanolone, furazabol, nandroloneoxandrolone, stanozolol, trenbolone acetates, dihydrotestosterone, 17-alpha-methyl-19-nortestosterone, fluoxymesterone, finasteride, turosteride, LY-191704, MK-306, betamethasone, betamethasonevalerate, cortisone, dexamethasone, dexamethasone 21 -phosphate, fludrocortisone, flumethasone, fluocinonide, fluocinonide desonide, fluocinolone, fluocinolone acetoneide, fluocortolone, halcinonide, halopredone, hydrocortisone, hydrocortisone 17-valerate, hydrocortisone 17-butyrate, hydrocortisone 21-acetates methyl prednisolone, prednisolone, prednisolone 21-phosphate, prednisone, triamcinolone, triamcinolone acetoneide, cortodoxone, fluoracetoneide, fludrocortisone, difluorsone diacetates, fluorandrenolone acetoneide, medrysone, arncinafel, amcinafide, betamethasone, chloroprednisone, clorcortelone, descinolone, desonide, dichlorisone, difluprednate, flucloronide, flumethasone, flumsolide, flucortolone, fluoromethalone, fluperolone, fluprednisolone, meprednisone, methyl meprednisolone, paramethasone, cortisone acetates, hydrocortisone cyclopentyl propionate, cortodoxone, flucetonide, fludrocortisone acetates, flurandrenolone acetoneide, medrysone, amcinafal, amcinafde, betamethasone, betamethasone benzoate, chloroprednisone acetates, clocortolone acetates, descinolone acetoneide, desoximetasone, dichlorisone acetates, difluprednate, flucloronide, flumethasone pivalate, flunisolide acetates, fluperolone acetates, fluprednisolone valerate, paramethasone acetates, prednisolamate, prednival, triamcinolone hexacetoneide, cortivazol, formocortal, nivazol, corticotrophin, thyrotropin, follicle stimulating hormone (FSH), luteinizing hormone (LH), gonadotrophin releasing hormone (GnRH), insulin, chloropropamides, glibenclamides, gliclazide, glipizide, tolazarnide, tolbutamides, and metformin, calcitonin, thyroxine, liothyronine, carbirnazole, propylthiouracil, octreotide, bromocriptine, clomiphene; diuretics, and antidiuretics, such as thiazide, bendrofluazide, chlorothiazide, chlorothalidone, dopamine, cyclopenthiazide, hydrochlorothiazide, indapamides, mefruside, meiycholthiazide, metolazone, quinethazone, bumetanide, ethacrynic acid, frusemide, spironolactone, amiloride, triamterene, desmopressin, lypressinan, vasopressin, ergometrine, oxytocin, gemeprost, prostaglandine, alprostadil (PGE1), prostacyclin (PGI2), dinoprost(prostaglandin f2-alpha), misoprostol; antimicrobial, antifungal, or antiseptic agents, such as cephalosporine, cephalexin, cefoxytin, cephalothin, penicilline, amoxycillin, amoxycillin+clavulanic acid, ampicillin, bacampicillin, benzathine penicillin, benzyl penicillin, carbenicillin, cloxacillin, methicillin, phenethicillin, phenoxymethyl penicillin, flueloxacillin, mezlocillin, piperacillin, ticarcillin, azlocillin, tetracycline, minocycline, chlorotetracycline, demeclocyeline, doxycycline, methaeycline, oxytetracycline, amikacin, gentamicin, kanamycin, neomycin, netilmicin, tobramycin, amorolfine, isoconazole, clotrimazole, econazole, miconazole, nystatin, terbinafine, bifonazole, amphotericin, griseofulvin, ketoconazole, fluconazole, flucytosine, fezatione, ticlatone, tolnaftate, triacetin, Zinc, pyrithione, sodium pyrithione, quinolone, nalidixie acid, cinoxacin, ciprofloxacin, enoxacin, norfloxacin, sulphonamides, phthalylsulphthiazole, sulfadoxine, sulphadiazine, sulphamethizole, sulphamethoxazole, sulphone, dapsone, chloramphenicol, clindamycin, erythromycin, erythromycin ethyl carbonate, erythromycin estolate, erythromycin glucepate, erythromycin ethyl succinate, erythromycin lactobionate, roxithromycin, lincomycin, natamycin, nitrofurantoin, spectinomycin, vancomycin, aztreonam, colistin IV, metronidazole, tinidazole, fusidic acid, trimethoprim, 2-thiopyridine n-oxidem, iodine-PVP complex, diiodohydroxyquine, hexachlorophene, chlorohexidine, chloroamine, benzoylperoxide, ethambutol, isoniazid, pyrazinamides, rifampicin, clofazimine, primaquine, pyrimethamine, chloroquine, hydroxychloroquine, quinine, mefloquine, halofantrine, acyclovir, acyclovir prodrugs, famciclovir, penciclovir, zidovudine, didanosinc, stavudine, lamivudine, zalcitabine, saquinavir, indinavir, ritonavir, n-docosanol, tromantadine, idoxuridine, mebendazole, thiabendazole, niclosamides, praziquantel, pyrantelembonate, diethyl carbamazine, plicamycin, cyclophosphamides, dacarbazine, fluorouracil, procarbazine, 6-mercaptopurine, mucophenolic acid, alcohols, ethanol, isopropanol, quaternary ammonium compounds, benzalkonium chloride, detrimide, cetypyridinium chloride, bisdequalinium diacetates, dequalinium chloride, chlorohexidine, chlorohexidine acetates, pMMB; chloramine, sodium dichloroiscyanuate, sodium hypochlorite; metabolistic agents, such as dexfenfluramine, fenfluramine diethyl propione, mazindol, phentermine, calcitriol, dihydrotachysterol; active ingredients for the immune system, such as meclozine, cyclizine, chlorocyclizine, hydroxyzine, brompheniramine, chloropheniramine, clemastine, cyproheptadine, dexchloropheniraraine, diphenhydramine, diphenylamine, doxylamine, mebhydrolin, mepyramine, phenirarnine, tripolidine, azatadine, diphenylpyraline, methdilazine, terfenadine, astemizole, loratidine, cetirizine; anesthetics, such as bupivacaine, amethocaine, lignocaine, cinchocaine, dibucaine, mepivacaine, prilocaine, etidocaine; neuromuscular blockers, such as suxamethonium, alcuronium, pancuronium, atracurium, gallamine, tubocurarine, vecuronium; smoke article replacements, such as nicotine, bupropione, ibogaine; dermatological agents, such as vitamin A, vitamin E, vitamin E acetate, vitamin E sorbate, vitamin D; allergens for desensibilization, for instance pollen or house-dust allergens; anti-acne agents such as isotretinoin, tretinoin, benzoyl peroxide; anti-psoriasis agents, such as etretinate, cyclosporin, calcipotriol; anti-itching agents, such as capsaicin or nonivamides; antiperspirant agents, such as methatropine nitrate, propantheline bromide, scopolamine, methscopolamine bromide; physiologically active peptides and proteins, for instance vasopressin or human growth hormone. Preferred are in particular antimycotics, such as, for instance, terbinafin, amorolfin, itraconazol, ciclopirox, or clotramizol. Also can be employed bexaroten, oxybutinin, or tolterodin.

Dosages for an active ingredient may be in the range from 0.01 ng to 50 g, in particular 0.01 mg to g, for instance 0.1 mg to 1 g or 1 mg to 300 mg per application.

As a physiologically compatible organic monomer/polymer for film-forming is, for instance, contemplated: acrylic acid, methacrylic acid, and derivatives, polyacrylate and polyacrylate derivatives, such as polybutyl methacrylate and polymethacrylic acid, polymethacrylate, ascorbyl palmitate, carbomer, carnauba wax, cellulose derivatives, such as cellulose acetate phthalate, Rosca mellose sodium, hydroxyethyl cellulose, hydroxypropyl cellulose, hydroxypropyl methyl cellulose, ethyl cellulose, hydroxypropyl methyl cellulose phthalate, hypromellose phthalate, crospovidon and its derivatives, cetyl alcohol and its derivatives, microcrystalline wax, poloxamer, polyethylene glycol, polyurethane, polyvinyl acetates, polyvinyl acetate phthalate, polyvinyl alcohol, povidone, silicone rubber and its derivatives, shellac, triglyceride derivatives. Also can be used derivatives, mixtures and/or copolymers of such polymers, in particular mixtures or copolymers of polyurethane or polyurethane derivatives and polymethacrylate or polymethacrylate derivatives. Optionally, at least one plasticizer for obtaining the required elasticity is contained. Examples of this are: polybutyl phthalate, benzyl benzoate, dibutyl sebacate, dimethyl phthalate, dibutyl phthalate, triacetin, glycol, and its derivatives, benzyl benzoate, glycerol, mineral oil, lanolin, alcohols (e.g., c8-alcohols), petroleum, lanolin alcohols, polyethylene glycol, sorbitol, triethylene citrate, propylene glycol, chlorobutanol, castor oil, and gelatin.

For making the composition, the used monomers/polymers may be used in the form of solutions. These solutions may in turn be aqueous and/or contain conventional, for instance the above mentioned organic solvents or may consist thereof.

As galenic excipients and/or dilution agents are contemplated all conventional excipients for transdermal systems, depending on the form of administration and/or the release rates. The amount and kind of the excipients should of course be compatible with the employed other component. Examples include: cosolvents, surfactants, emulgators, antioxidants, preservation agents, stabilizers. Examples of galenic excipients are paraffin oils, isopropyl esters, myristates, ethanol, silicone oils, and oils for instance obtained from olives, peanuts, or sesame. As surfactants are contemplated ethoxylated fatty acids, glycerol monostearate, phosphate esters, and other conventional emulgators and surfactants. An example of a preservation agent is hydroxybenzoate ester. Typical cosolvents and adjuvants are ethanol, propanol, isopropanol, acetone, dimethyl ether, glycol ether, such as diethylene glycol monoethyl ether. These excipients should of course be compatible with the requirement that the composition after application on the skin or a transfer film will form a contact-dry film in a sufficiently short time, typically 10 s to 10 min.

The pharmaceutical composition may be applied in an arbitrary way on the skin or on a substrate. This includes spreading, brushing, raking, or spraying.

The pharmaceutical composition may be prepared in an aerosol dispenser for spraying the composition on the skin of a human or of a animal. If applicable, the composition also contains a propellant, if the aerosol dispenser is to operate under pressure.

The invention relates in so far also to a pharmaceutical composition for the dermal and/or transdermal application, containing at least one compound of Formula I or a mixture of different such compounds, at least one dermally acting and/or transdermally resorbing pharmaceutical active ingredient or a mixture of different such active ingredients, optionally at least one film-forming organic monomer or polymer or a mixture of such monomers and/or polymers, optionally at least one aqueous or additional organic solvent or a mixture of different such additional solvents, optionally at least one galenic excipient or a mixture of different such excipients, optionally at least one dermo-cosmetically acting additive or a mixture of different such additives.

The invention further comprises an aerosol dispenser with a pharmaceutical composition according to the invention. In principle, all conventional aerosol dispensers can be used, wherein the term aerosol also comprises spray mists.

A particularly suitable aerosol dispenser for the application of transdermally acting pharmaceutical active ingredients comprises a vessel containing the pharmaceutical active ingredient in a liquid solution, the interior of said vessel being pressurized by means of a driving gas or the interior of which can be pressurized by means of a pump device, an atomizing device connected to the interior of the vessel, an operable delivery valve arranged between the interior of the vessel and the atomizing device, and a spray head connected to the delivery valve, wherein optionally a head portion having a vessel opening is provided, which can be mounted on the container, and wherein the head portion includes an application opening with a mounting lip for placing the head portion on the skin. In the embodiment with the head portion, it is achieved that when operating the delivery valve, the interior of the head portion is filled with the aerosol or spray mist forming the patch, wherein the atomizing device will direct the aerosol jet preferably in the direction of the mounting lip, i.e., the atomizing device has an outlet opening that shows in the direction of the mounting lip. By that the head portion with the vessel opening practically tightly seals against the vessel and with the mounting lip against the skin, the aerosol cannot exit this interior of the head portion. A contamination of adjacent skin regions, of clothing, third persons, or the surroundings cannot occur. In particular, an undesired inhalation of the aerosols is excluded. Further, by the edge of the mounting lip, a patch with a defined perimeter is formed, wherein further the thickness of the patch is nearly homogeneous over the thus formed surface of the patch. Hereby, a particularly well defined kinetics of the delivery of active ingredient to the skin and thus a very well controllable bioavailability of the active ingredient is achieved. In principle, the head portion can be made of an arbitrary medically compatible material. Preferred is an organic polymer, for instance a thermoplastic material, for instance polyolefins such as PE or PP, or a thermoplastic elastomer, as indicated below. It is preferred, when at least the mounting lip or the head portion is totally formed of one or more different rubber-elastic materials. These include, for instance, natural rubber, modified natural rubber, silicone rubber, EPDM, thermoplastic elastomers on olefin basis, such as PP/EPDM, thermoplastic elastomers on urethane basis, thermoplastic polyester elastomers or copolyesters, styrene block polymers such as SBS, SEBS, SEPS, SEEPS, or MBS, and thermoplastic copolyamides, such as PEBAX. The head portion may have, in the area of the mounting lip, a shape, which is suitable for a complete rest on a target area on the skin. In principle, many such shapes are conceivable, suitably however the configuration should have a circular or oval annular cross-section. Preferably, the cross-sectional area of the mounting lip is larger than the cross-sectional area of the vessel opening, i.e., the head portion widens from the vessel opening to the mounting lip. The head portion may be configured with a substantially cone-shaped mantle surface having, in the cross-section in the plane of the center axis of the cone, a straight, convex, or concave cone surface. Suitably, the head portion has a lower hardness in the area of the mounting lip than in the area outside the mounting lip. The mounting lip may, for instance, have a Shore A hardness of up to 10, whereas the head portion otherwise may have a Shore A hardness of 20, in particular 40, up to 120 and more. The head portion may be solidly or reversibly connectable to the vessel. In the first case, the head portion is connected, when manufacturing the aerosol dispenser, to the vessel by positive, non-positive, or bonded fitting. Then, it cannot be removed without destruction. In the latter case, vessel and head portion may be provided separately for the user, who will then place the head portion, for instance by a clamped connection or snap connection, on the vessel and, if applicable, also removes it again, for instance to permit a space-saving storage of the aerosol dispenser. If the atomizing device comprises an outlet opening, which shows in the direction of the mounting lip, it is secured that the main portion of the aerosol will arrive on the skin and only a smaller portion will deposit on the inner wall of the head portion and is thus lost. The cross-sections of the vessel opening and of the application opening may be oriented in parallel to each other or angled to each other, i.e., the head portion may also include an angular piece in the area of the vessel opening.

The invention further relates to a dermal or transdermal patch for adhering on the skin of a human or an animal, obtainable by spraying or applying a pharmaceutical composition according to the invention on a substrate film (for instance a transfer film).

A dermal or transdermal patch can be achieved on the skin of a human or an animal by that a pharmaceutical composition according to the invention is applied or sprayed on the skin.

A pharmaceutical composition according to the invention can be made by that at least one dermally acting and/or transdermally resorbing pharmaceutical active ingredient is mixed in a pharmacologically effective amount with at least one compound of Formula I and optionally with at least one film-forming organic monomer or polymer, at least one aqueous or additional organic solvent, at least one galenic excipient, and/or at least one dermo-cosmetically acting additive.

One aspect of independent significance of the invention relates to the use of the compound of Formula I in other forms of administration than in dermal/transdermal systems, for instance in solutions, which are applied on body-own mucous membranes and/or other inner walls of the body. Thus, for instance, it is possible to formulate pharmaceutical substances common in veterinary medicine for treating mastitis of cows in a high amount in milk, in particular solubilize them, when the compound of Formula I is admixed, for instance in an amount of up to 50 vol.-%, in particular of 10 to 45 vol.-%, preferably of 20 to 40 vol.-%, of the obtained mixture. The thus treated milk, in turn, can then be applied to the udder, where the active ingredient then takes its healing effect. These active ingredients may however also be delivered, as described above, transdermally by application of a transdermal composition on the udder.

The explanations with regard to the uses and components according to the invention apply in a corresponding manner to all above aspects of the invention.

In the following, the invention will be explained in more detail with respect to embodiments representing examples only.

### EXAMPLE 1

### Solubilities of Various Active Ingredient Classes in Carvacrol or Carvacrol-Containing Formulations

The solubilities of the active ingredients listed in Table I and being usable for the purposes of the invention in carvacrol or carvacrol-containing formulations were measured. The solubilities are overall considerably higher than the solubilities of the same active ingredients in other solvents common for transdermal systems. The mixing ratios are always given as volume proportions. Liqui-Patch® are formulations of ready-to-use mixtures of polymers and other additives for making transdermal systems and are obtainable from the company epinamics GmbH. In the formulation employed here, Liqui-Patch® contains the following components: mixture of 10 g triethyl citrate, 100 g water (demineralized or medical), 100 g DynamX®, 790 g ethanol. The following elements of the table using Liqui-Patch and in mixture with carvacrol are consequently also examples of preparations according to the invention. Similar results are also obtained, for instance, with the isomer thymol, as can be seen in some elements of the table.

TABLE I
Solubilities of active ingredients in
carvacrolor thymol preparations.

| | | Dissolved | |
|---|---|---|---|
| Active ingredient | Solvent | amount | |
| Lidocaine HCl | Carvacrol | 260 | mg/ml |
| Testosterone | Carvacrol | 220 | mg/ml |
| Erythromycin | Carvacrol | 110 | mg/ml |
| Uric acid | Carvacrol | <20 | mg/ml |
| 8-Hydroxyquinoline | Carvacrol | >100 | mg/ml |
| 6-Aminouracil | Carvacrol | <50 | mg/ml |
| Coumarin | Carvacrol | >200 | mg/ml |
| Rutin | Carvacrol/ | 20 | mg/ml |
| | Methanol (1:1) | | |
| Thymoquinone | Carvacrol | >100 | mg/ml |
| Curcumin | Carvacrol | 20 | mg/ml |
| Nalidixic Acid | Carvacrol | 108 | mg/ml |
| Salicylic acid | Carvacrol/ | 240 | mg/ml |
| | Methanol (1:1) | | |
| Sulfamethoxazole | Carvacrol | 50 | mg/ml |
| Bexarotene | Carvacrol | 60 | mg/ml |
| Bexarotene | Carvacrol/ | 30 | mg/ml |
| | Liqui-Patch | | |
| | (1:1) | | |
| Lauric acid | Carvacrol | 500 | mg/ml |
| Lauric acid | Carvacrol/ | 250 | mg/ml |
| | Liqui-Patch | | |
| | (1:1) | | |
| Terbinafine | Thymol/Ethanol | 406 | mg/ml |
| | (1:1) | | |
| Granisetron | Carvacrol 62.5% | 71.25 | mg/ml |
| | Methanol 31.25% | | |
| | 2M NaOH/H₂O | | |
| | 6.25% | | |
| Clotrimazole | Carvacrol | 410 | mg/ml |
| Haloperidol | Carvacrol | 100 | mg/ml |
| Flunarizine*2HCl | Carvacrol | 60 | mg/ml |
| Cromoglicic acid-Na | Carvacrol | 50 | mg/ml |
| Itraconazole | Carvacrol | 300 | mg/ml |
| | | (after | |
| | | heating) | |
| Ketoconazole | Carvacrol | 140 | mg/ml |
| Ciprofloxacin | Carvacrol | 80 | mg/ml |
| Clotrimazole | Carvacrol | 410 | mg/ml |
| 5-Fluorouracil | Carvacrol | 25 | mg/ml |
| Ellagic acid | Carvacrol | 60 | mg/ml |
| Protoporphyrin IX | Carvacrol | 64 | mg/ml |
| Cromoglicic acid | Carvacrol | 50 | mg/ml |
| disodium salt | | | |

### EXAMPLE 2

### Solubilities of Two Exemplary Active Ingredients in Different Solvents

In Table II, the solubilities in mg/ml of clotrimazole and testosterone are shown. It can be seen the comparatively considerably higher solubilities in solvents with compounds employed according to the invention.

“LP” refers to Liqui-Patch®.

TABLE IIa

| | Solubility | Solubility |
|---|---|---|
| Solvent | Clotrimazole | Testosterone |
| Ethanol | 70 mg/ml | 90 mg/ml |
| carvacrol | 410 mg/ml | 230 mg/ml |
| 20% Thymol/80% EtOH | 230 mg/ml | 330 mg/ml |
| 20% Carvacrol/80% | 240 mg/ml | 260 mg/ml |
| EtOH | | |
| Guaiacol | 250 mg/ml | 270 mg/ml |
| 20% Guaiacol/80% | 260 mg/ml | 300 mg/ml |
| EtOH | | |
| 20% Guaiacol/80% LP | 270 mg/ml | 270 mg/ml |
| Acetone | 110 mg/ml | 60 mg/ml |
| 20% Thymol/80% | 240 mg/ml | 180 mg/ml |
| Acetone | | |
| 20% Carvacrol/80% | 240 mg/ml | 170 mg/ml |
| Acetone | | |
| LP | 50 mg/ml | 190 mg/ml |
| 20% Thymol/80% LP | 250 mg/ml | 300 mg/ml |
| 20% Carvacrol/80% | 240 mg/ml | 250 mg/ml |
| LP | | |

TABLE IIb

| | Solubility |
|---|---|
| Solvent | Testosterone |
| Ethanol | 90 mg/ml |
| Anisole | <100 mg/ml |
| 4-Methyl-Anisole | <250 mg/ml |
| Methyl salicylate (4MS) | <100 mg/ml |
| 20% Anisole/80% EtOH | 500 mg/ml |
| 20% 4MS/80% EtOH | 335 mg/ml |
| p-Cymene | insoluble (<100) |
| 20% p-Cymene/80% Ethanol | 300 |
| Gamma-Terpinene | insoluble (<100) |
| 20% Gamma-Terpinene/ | 200 |
| 80% ethalol | |
| (S)-(—)-Limonene | insoluble (<100) |
| 20% (S)-(—)-Limonene/80% | 200 |
| Ethanol | |

### EXAMPLE 3

### Volatility of Carvacrol

For testing whether carvacrol is suitable for transdermal systems or whether carvacrol is contained sufficiently long in transdermal systems, the volatility was investigated with different admixtures. The volatility is important, in order to keep the active ingredients sufficiently long in solution in the film.

For the measurement, two spots of 5 μl of each formulation, containing 1 mg carvacrol, were applied per point of time on a glass plate kept at 35° C. Two spots of a respective point of time (30 min. and 3 h after application of the spot) were wiped off with a cotton applicator. The amount of wiped-off carvacrol was determined. The difference to 100% intended recovery rate is the volatile carvacrol. The results are shown in Table III. The abbreviation V. refers to “volatile”, the abbreviation C. refers to carvacrol. The share of carvacrol is always 20%, the remainder relative to the sum of carvacrol and the additive is ethanol. Amounts are given in vol.-%.

TABLE III
Volatility of carvacrol.

| Additive | V.C. 30 min. | V.C. 3 h |
|---|---|---|
| Isopropyl myristate 10% | 26% | 46% |
| Isopropyl myristate 20% | 24% | 32% |
| Ethylmethyl ketone 10% | 33% | 100% |
| Ethylmethyl ketone 20% | 45% | 100% |
| Propylene glycol 10%/ | 40% | 97% |
| menthol 5% | | |
| Propylene glycol 20%/ | 0% | 46% |
| menthol 10% | | |
| Water 10% | 18% | 94% |
| Water 20% | 0% | 94% |
| Propylene carbonate 10% | 29% | 49% |
| Propylene carbonate 20% | 11% | 24% |
| Glycerol 10% | 28% | 96% |
| Glycerol 20% | 33% | 88% |
| Propylene glycol 10% | 40% | 51% |
| Propylene glycol 20% | 1% | 33% |

It can be seen that the volatility of carvacrol is reduced in an advantageous manner, for instance, by propylene carbonate, propylene glycol, or isopropyl myristate.

1. A method of solubilizing a pharmaceutical active ingredient in an aqueous and/or organic solvent, the solvent being a compound of Formula I or a mixture of different such compounds, or a mixture of the compound or of the mixture of the different such compounds with an additional solvent, comprising the step of solubilizing the active ingredient in the compound or in the solvent containing the compound, 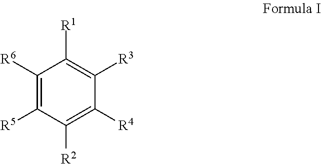
wherein R1 to R5 are independently from each other, identically or different, selected from the group consisting of —H, —OH, methyl, ethyl, propyl, isopropyl, isopropenyl, and alkoxy, wherein R6 is selected from —O—R7 and —CO—R7, with R7 being selected from —H, C1-C3-alkyl, C6-aryl and C7-C10-aralkyl, substituted or not substituted, and wherein the aromatic ring of the illustration of Formula I may be replaced by C6-cycloalkyl, saturated or mono-unsaturated or di-unsaturated, at an arbitrary position of the cycloalkyl ring.

2. The method according to claim 1, wherein R1 is methyl or methoxy, R2 is isopropyl, isopropenyl, or —H and R3 to R5 are —H, or R1 is isopropyl, R2 is methyl, and R3 to R5 are —H.

3. A use of the method according to claim 1 for making a pharmaceutical composition in the form of a dermal or transdermal preparation, wherein the pharmaceutical active ingredient is solubilized in a pharmacologically effective amount in the solvent.

4. The use according to claim 3, wherein the pharmaceutical composition contains the following components:
a) at least one compound of Formula I or a mixture of different such compounds,
b) at least one dermally acting and/or transdermally resorbing pharmaceutical active ingredient or a mixture of different such active ingredients,
c) optionally at least one physiologically compatible film-forming monomer or polymer or a mixture of different such monomers and/or polymers,
d) optionally at least one aqueous or additional organic solvent or a mixture of different such additional solvents,
e) optionally at least one galenic excipient or a mixture of different such excipients,
f) optionally at least one dermo-cosmetically acting additive or a mixture of different such additives.

5. The use according to claim 3, wherein the pharmaceutical composition is prepared for spraying on the skin.

6. The use according to claim 3, wherein the additional organic solvent is selected from the group consisting of methanol, ethanol, butanol, 1-propanol, 2-propanol, 1-bromopropane, 1-octanol, dimethyl ether, diethyl ether, methyl ethyl ether, benzene, chloroform, DMSO, acetoneitril, acetone, dioxane, ethylene glycol, propylene glycol, dimethyl formamide, benzene, ethyl acetate, PEG400, hexane, trichloroethylene, ethyl catate, N-methyl-2-pyrrolidone, toluene, DMSO, DMF, pyridine, isopropyl myristate, dichloromethane, 1,4-dioxane, propylene carbonate, and aniline, or mixtures of such solvents, in particular selected from additional solvents inhibiting the volatility of the compound, such as propylene carbonate, propylene glycol, isopropyl myristate.

7. The use according to claim 3, wherein the pharmaceutical composition is prepared in an aerosol dispenser for spraying the composition on the skin of a human or of an animal.

8. A pharmaceutical composition for dermal and/or transdermal application containing at least one compound of Formula I or a mixture of different such compounds, at least one dermally acting and/or transdermally resorbing pharmaceutical active ingredient or a mixture of different such active ingredients, optionally at least one film-forming organic monomer or polymer or a mixture of different such monomers and/or polymers, optionally at least one aqueous or additional organic solvent or a mixture of different such additional solvents, optionally at least one galenic excipient or a mixture of different such excipients, optionally at least one dermo-cosmetically acting additive or a mixture of different such additives.

9. An aerosol dispenser with a pharmaceutical composition according to claim 8.

10. A dermal or transdermal patch to adhere on the skin of a human or an animal, obtainable by spraying or applying a pharmaceutical composition according to claim 8 on a substrate film.

11. A method of making a dermal or transdermal patch on the skin of a human or an animal, comprising the step of applying or spraying a pharmaceutical composition according to claim 8 on the skin.

12. A method of making a pharmaceutical composition according to claim 8, comprising the step of mixing at least one dermally acting and/or transdermally resorbing pharmaceutical active ingredient in a pharmacologically effective amount with at least one compound of Formula I and optionally with at least one aqueous or additional organic solvent, at least one galenic excipient and/or at least one dermo-cosmetically acting additive, respectively.
